(19) United States
(12) Patent Application Publication
(10) Pub. No.: US 2007/0148127 A1
(43) Pub. Date: Jun. 28, 2007

(54) ANTIMICROBIAL OPHTHALMIC TREATMENT SYSTEM AND METHOD

(76) Inventors: David J. Heiler, Avon, NY (US); Joseph C. Salamone, Boca Raton, FL (US); Richard V. Smerbeck, Pittsford, NY (US); Matthew S. Jonasse, Webster, NY (US)

Correspondence Address: Bausch & Lomb Incorporated, One Bausch & Lomb Place, Rochester, NY, 14604-2701, US

(21) Appl. No.: 11/611,369

(22) Filed: Dec. 15, 2006

Related U.S. Application Data
(60) Provisional application No. 60/753,020, filed on Dec. 22, 2005.

(51) Int. Cl. A61K 31/785; A61K 31/155

(52) U.S. Cl. 424/78.27; 514/635

(57) ABSTRACT

An ophthalmic treatment system comprises an ophthalmic treatment solution and a biguanide antimicrobial agent in solid form and in contact with said treatment solution such as to inhibit bacterial and/or fungal growth in said solution. The biguanide is partially or wholly terminated with an amine compound.

### CROSS REFERENCE

This application claims the benefit of Provisional Patent Application No. 60/753,020 filed Dec. 22, 2005 and is incorporated herein by reference.

### FIELD

The present invention relates to ophthalmic treatment system and method that maintains the sterility of the treatment solution during storage and dispensing.

### BACKGROUND

The contact of eye tissue with fungi, bacteria and other microorganisms may lead to a variety of serious eye infections, such as microbial keratitis. The contact of eye tissue with bacteria may result when an ophthalmic solution contaminated with bacteria is instilled directly in the eye. Examples of such ophthalmic solutions distilled directly in the eye are eye drop solutions (for example, for treating dry eye) or contact lens drop solutions (for example, for wetting a contact lens while worn). Additionally, eye tissue may be contacted with bacteria by placing a contact lens on the eye where the contact lens is contaminated with bacteria. The risk of eye infection is increased when bacteria is adhered to a contact lens, since the bacteria may remain in contact with the eye tissue for a prolonged period of time.

For this reason, ophthalmic treatment compositions, such as eye drops and contact lens treating solutions, conventionally include an antimicrobial agent that acts as a preservative to inhibit the growth of bacteria, as well as other infectious organisms, in case the solution becomes contaminated with such organisms. For contact lens treating solutions, the antimicrobial agent used to preserve the solution may also serve to disinfect contact lenses when rinsed or soaked with the solution. Various antimicrobial agents are known for use as preservatives in ophthalmic compositions. Such antimicrobial agents should have a broad spectrum of antimicrobial activity but must also be non-irritating to the eye.

For example, U.S. Pat. No. 5,741,886 discloses a linear polymeric biguanide or a salt thereof wherein the polymeric chain is wholly or substantially wholly terminated at its cyanoguanidine end group by a primary or secondary monoamine containing 2-90 carbon atoms, said monoamine being optionally substituted with a functionality selected from the group consisting of fluoride, chloride, bromide, iodide, nitro, hydroxyl, ether, sulfide, disulfide, sulfoxide and sulfone. Preferred monoamines include n-dodecylamine, 2-aminothiazole, 2-aminobenzotriazole, polyoxyethylene monoamine, polyoxypropylene monoamine and polyoxy(ethylene/propylene) monoamine. The resultant polybiguanide is said to be useful as the active ingredient in fungicidal or bactericidal compositions, especially ophthalmic compositions, such as wetting solutions, enzyme cleaners, sterile saline solutions, preservatives for ophthalmological preparations and disinfectant solutions.

However, many antimicrobial agents have a tendency to irritate eye tissue, especially at higher concentrations. Therefore, it is generally advantageous to employ as low as possible concentration of antimicrobial agent to avoid the risk of eye irritation.

U.S. Published Patent Application No. 2004/0063620, published Apr. 1, 2004, discloses an ophthalmic composition comprising a primary antimicrobial agent, such as sorbic acid or a biguanide, and a polycation material that enhances the antimicrobial efficacy of said primary antimicrobial agent against bacteria, such as E. coli. Preferred polycation materials are cationic cellulose derivatives.

One alternative solution to the problem of contamination that avoids the need for preservatives is the single dose dispenser. Such dispensers, however, are made only for one time use, and then are discarded, adding considerably to packaging costs and waste. Moreover, more sterile solution than is required for a single dose usually is packaged which adds to the expense of the treatment. Another problem is that persons may attempt to use the single dose dispenser multiple times, which can result in contamination of the liquid being dispensed.

Another alternative solution is to provide packaging with antimicrobial protection. For example, U.S. Pat. No. 5,611,464 discloses a container for dispensing an ophthalmic composition comprising a strong preservative and a weak preservative wherein the container has a dispensing tip including a media which will inactivate or remove the strong preservative when the composition passes through the dispensing tip without completely removing said weak preservative. The weak preservative inhibits microbial growth in the tip media, while the strong preservative inhibits microbial growth in the composition or inhibits degradation of an active agent contained in the composition. The strong preservative can be poly(hexamethylene biguanide)

U.S. Pat. No. 5,869,073 describes a dispenser for dispensing a sterile liquid wherein the dispenser comprises a non-leaching antimicrobial element for inhibiting microbial contamination of the sterile liquid. The antimicrobial element can comprise a filter or filters attached to a nozzle assembly of the dispenser and positioned across a liquid dispensing passageway in the nozzle assembly. Alternately, the dispenser may contain an antimicrobial element disposed within the body of the container such that it remains in contact with the sterile solution at all times, e.g., during storage and dispensing. This is accomplished by providing a substrate having permanently attached thereto or coated thereon an antimicrobial agent. The substrate may be a bead or plurality of beads, a membrane, cartridge, filter, wool, cotton, baffle or fibrous bundle, for example. The element may be free-floating in the solution within the container or may be attached to or immobilized within the container. In this embodiment, the antimicrobial element remains in contact with the solution thereby insuring its sterility even after repeated doses have been dispensed by a user. Preferred antimicrobial agents include chlorhexidine or polyhexamethylene biguanide which may be modified to permit direct immobilization on a metallic or non-metallic surface.

U.S. Pat. No. 6,579,539 discloses an article, such as a medical article, that resists microbial infection. The article is provided on at least one of its surfaces with a coating comprising a metallic silver layer and a polyether polyurethaneurea block copolymer layer; wherein the block copolymer layer contains an antimicrobial agent capable of diffusing from the copolymer layer. In a preferred embodiment, the antimicrobial agent is a biguanide.

U.S. Published Patent Application No. 2003/0159983, published Aug. 28, 2003, discloses an antimicrobial porous component formed of polymeric material having grafted on its surface and in bulk ammonium units with antimicrobial and/or bactericidal and/or antifungal activity. The porous component is arranged to have a porosity that is sufficiently high enough to allow the passage through its structure of a liquid of compatible viscosity, and, on the other hand, sufficiently low to ensure that contaminant microorganisms are trapped within its structure. The porous component conveniently forms a porous plug in the outlet orifice of a liquid dispenser containing, for example, an ophthalmic solution.

Despite the foregoing advances, there is a continuing need for ophthalmic treatment systems that combine enhanced antimicrobial preservative activity with reduced tendency for eye irritation.

### SUMMARY

In one aspect, the present invention resides in ophthalmic treatment system comprising an ophthalmic treatment solution and a biguanide antimicrobial agent in solid form and in contact with said treatment solution such as to inhibit bacterial and/or fungal growth in said solution, wherein said biguanide is partially or wholly terminated with an amine compound.

Preferably, said biguanide antimicrobial agent is substantially insoluble in said solution.

In one embodiment, the system includes a container within which said ophthalmic treatment solution and said biguanide antimicrobial agent are accommodated. Conveniently, the biguanide antimicrobial agent is incorporated in one or more elements separate from said container. Alternately, the biguanide antimicrobial agent is bonded to the internal surface of the container.

Conveniently, the biguanide is partially or wholly terminated at its cyanoguanidine end group by a primary or secondary monoamine containing about 2 to about 90 carbon atoms and optionally is substituted with a functionality selected from the group consisting of fluoride, chloride, bromide, iodide, nitro, hydroxyl, ether, sulfide, disulfide, sulfoxide and sulfone.

The invention further includes a method of rendering an ophthalmic treatment solution resistant to bacterial and/or fungal growth comprising contacting the solution with a heterogeneous phase composition comprising a biguanide antimicrobial agent wherein said biguanide is partially or wholly terminated with an amine compound.

### DETAILED DESCRIPTION

As used herein, the term “ophthalmic composition” denotes a composition intended for application in the eye or intended for treating a medical device to be placed in contact with the eye, such as a contact lens. Ophthalmic compositions specifically include compositions for direct instillation in the eye, including eye drop solutions such as for treating dry eye, and contact lens treating solutions distilled directly in the eye such as for rewetting a contact lens while worn. Ophthalmic compositions also include compositions instilled indirectly in the eye, such as contact lens treating solutions for treating the contact lens prior to the lens being inserted on the eye, as well as contact lens multi-purpose solutions.

The term “preservative” or like terms denotes agents included in the ophthalmic compositions for the purpose of inhibiting the growth of microorganisms in the product, thereby helping to maintain sterility of the composition. The term “antimicrobial agent” denotes the specific active agent which provides the antimicrobial efficacy.

In the case of contact lens treating solutions, the methods and/or compositions of the present invention may be applicable to the conventional contact lens categories: (1) hard lenses formed from materials prepared by polymerization of acrylic esters, such as polymethyl methacrylate (PMMA), (2) rigid gas permeable (RGP) lenses formed from silicone acrylates and fluorosilicone methacrylates, (3) soft, hydrogel lenses, and (4) non-hydrogel elastomer lenses.

As an example, soft hydrogel contact lenses are made of a hydrogel polymeric material, a hydrogel being defined as a cross-linked polymeric system containing water in an equilibrium state. In general, hydrogels exhibit excellent biocompatibility properties, i.e., the property of being biologically or biochemically compatible by not producing a toxic, injurious or immunological response in a living tissue. Representative conventional hydrogel contact lens materials are made by polymerizing a monomer mixture comprising at least one hydrophilic monomer, such as (meth)acrylic acid, 2-hydroxyethyl methacrylate (HEMA), glyceryl methacrylate, N,N-dimethacrylamide, and N-vinylpyrrolidone (NVP). In the case of silicone hydrogels, the monomer mixture from which the copolymer is prepared further includes a silicone-containing monomer, in addition to the hydrophilic monomer. Generally, the monomer mixture will include a crosslinking monomer, i.e., a monomer having at least two polymerizable radicals, such as ethylene glycol dimethacrylate, tetraethylene glycol dimethacrylate, and methacryloxyethyl vinylcarbonate. Alternately, either the silicone-containing monomer or the hydrophilic monomer may function as a crosslinking agent.

In general, the present invention is directed to an ophthalmic treatment system comprising an ophthalmic treatment solution and an antimicrobial agent in solid form and in contact with said treatment solution such as to inhibit bacterial and/or fungal growth in said solution. The treatment solution is preservative free other than the antimicrobial agent and can be an eye drop solution or a contact lens wetting, cleaning, disinfecting, conditioning or multipurpose solution.

The antimicrobial agent is a biguanide and in particular a linear polymeric biguanide or salt thereof wherein the polymeric chain is wholly or substantially wholly terminated at its cyanoguanidine end group by a primary or secondary monoamine containing about 2 to about 90 carbon atoms, preferably about 2 to about 20 carbon atoms. The monoamine is optionally substituted with a functionality selected from the group consisting of fluoride, chloride, bromide, iodide, nitro, hydroxyl, ether, sulfide, disulfide, sulfoxide and sulfone.

Conveniently, the monoamine is an aliphatic, cycloaliphatic, heterocyclic, aryl, aralkyl, alkaryl or polyether monoamine containing oxyalkylene groups such as oxyethylene and/or oxypropylene. More preferably, the monoamine is a primary amine containing 3 to 70 carbon atoms, e.g. n-dodecylamine, 2-aminothiazole, 2-amino-benzothiazole, polyoxyethylene monoamine, polyoxypropylene monoamine or polyoxy(ethylene/propylene) monoamine. Preferably, the monoamine is octadecylamine.

Typically the polymeric biguanide has recurring units of the general formula:

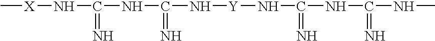

where the organic radicals represented by X and Y are the same or different organic bridging groups and conveniently are the same or different C₂ to C₁₄₀ aliphatic, cycloaliphatic, heterocyclic, aryl, alkaryl, aralkyl or oxyalkylene radicals. In one preferred embodiment, at least one of X and Y is a C₄ to C₁₆ polymethylene radical. One suitable bigaunide is polyhexamethylene biguanide (PHMB).

The end-capped polybiguanides of the present invention are readily prepared by a variety of multi-step processes wherein the linear, polymeric biguanide intermediates are prepared and isolated and thereafter reacted with the monoamine end-group modifier.

In one type of process, the polybiguanide intermediate is prepared by reacting equimolar amounts of the desired diamine (the diamine may be one diamine or a mixture of diamines, as desired) and one mole of the desired biscyanoguanidine compound or a mixture of the desired biscyanoguanidine compounds in the presence of hydrochloric acid. The components are conveniently brought into reaction by heating them alone or in a neutral solvent, e.g. water, alcohols, glycols, glycol ethers, etc. at a temperature of 90 to 190° C., preferably 100 to 170° C., for a period of 2-24 hours, preferably 2-12 hours.

Another method for preparing the polybiguanide intermediate involves the reaction of one mole of sodium dicyanamide and 0.5 mole of the desired diamine (one diamine or a mixture of diamines, as desired) in the form of its dihydrochloride salt. The reaction mixture is heated as is or in one of the solvents mentioned above at a temperature of 90 to 190° C., preferably 100 to 170° C., for 2-12 hours. The resultant 0.5 mole of the biscyanoguanidine compound is separated from the sodium chloride and thereafter reacted with 0.5 mole of the same or different diamine dihydrochloride as is or in one of the solvents mentioned above at a temperature of 90 to 190° C., preferably 100 to 170° C., for a period of 2-24 hours, preferably 2-12 hours.

The processes mentioned above for preparation of the linear, polymeric biguanide intermediates are well known in the prior art, e.g. see U.S. Pat. Nos. 2,643,232, 3,428,576 and 4,891,423 and British Patent Nos. 702,268, 1,152,243 and 1,167,249. In addition, polyhexamethylene biguanide is commercially available from Zeneca Biocides, Inc. of Wilmington, Del. as a 20% aqueous solution under the trade name COSMOCL-CQ.

The end-capped polybiguanides of the invention are readily prepared by the reaction of the desired polybiguanide with the desired primary or secondary monoamine. In this manner, only the cyanoguanidine end group is modified (i.e. “end-capped”). This type of modification of principally poly-condensation polymers is documented in the literature and is known to those skilled in polymer chemistry.

The end-capped polyhexamethylene biguanides achieve a higher level and rate of kill of a broad spectrum of pathogenic compositions while having a low level of toxicity.

In the ophthalmic treatment system of the invention the biguanide antimicrobial agent is insoluble in the ophthalmic treatment solution and is immobilized in contact with the treatment solution so as inhibit bacterial and/or fungal growth in said solution substantially without being leached into said solution. In one embodiment, the end-capped biguanide is immobilized on part or all of the internal surface of a container, such as a plastic bottle, which accommodates the treatment solution. In another embodiment, the biguanide is immobilized on one or more elements, such as beads, discs or a fibrous material, contained within, but separate from, the container. The end-capped biguanide may be coated on a substrate, such as the container walls, or attached to the substrate by covalent bonding, ionic interaction, coulombic interaction, hydrogen bonding or interpenetrating networks.

While the present invention has been described and illustrated by reference to particular embodiments, those of ordinary skill in the art will appreciate that the invention lends itself to variations not necessarily illustrated herein. For this reason, then, reference should be made solely to the appended claims for purposes of determining the true scope of the present invention.

What is claimed is:

1. An ophthalmic treatment system comprising an ophthalmic treatment solution and a biguanide antimicrobial agent in solid form and in contact with said treatment solution such as to inhibit bacterial and/or fungal growth in said solution, wherein said biguanide is partially or wholly terminated with an amine compound.

2. The system of claim 1 wherein said biguanide antimicrobial agent is immobilized on a solid substrate.

3. The system of claim 1 or claim 2 wherein said biguanide antimicrobial agent is substantially insoluble in said solution.

4. The system of any preceding claim wherein said ophthalmic treatment solution is selected from an eye drop, a contact lens wetting solution, a contact lens cleaning solution, and a contact lens multi-purpose solution.

5. The system of any preceding claim and including a container within which said ophthalmic treatment solution and said biguanide antimicrobial agent are accommodated.

6. The system of claim 5 wherein said biguanide antimicrobial agent is incorporated in one or more elements contained within, but separate from, said container.

7. The system of claim 5 wherein said biguanide antimicrobial agent is provided on the internal surface of the container.

8. The system of any preceding claim wherein said biguanide is partially or wholly terminated at its cyanoguanidine end group by a primary or secondary monoamine containing about 2 to about 90 carbon atoms.

9. The system of claim 8 wherein said monoamine is substituted with a functionality selected from the group consisting of fluoride, chloride, bromide, iodide, nitro, hydroxyl, ether, sulfide, disulfide, sulfoxide and sulfone.

10. The system of claim 9 wherein said functionality is chloride.

11. The system of any one of claims 8 to 10 wherein said monoamine includes octadecylamine.

12. The system of any preceding claim wherein said biguanide is a polymeric biguanide having recurring units of the general formula: 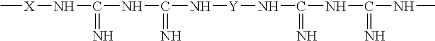
where the organic radicals represented by X and Y are the same or different organic bridging groups and conveniently are the same or different C₂ to C₁₄₀ aliphatic, cycloaliphatic, heterocyclic, aryl, alkaryl, aralkyl or oxyalkylene radicals.

13. The system of claim 12 wherein at least one of X and Y is a C₄ to C₁₆ polymethylene radical.

14. The system of any preceding claim wherein said biguanide is polyhexamethylene biguanide.

15. A method of rendering an ophthalmic treatment solution resistant to bacterial and/or fungal growth comprising contacting the solution with a heterogeneous phase composition insoluble in said solution and comprising a biguanide antimicrobial agent wherein said biguanide is partially or wholly terminated with an amine compound.

16. The method of claim 15 wherein said biguanide is partially or wholly terminated at its cyanoguanidine end group by a primary or secondary monoamine containing about 2 to about 90 carbon atoms.

17. The method of claim 16 wherein said monoamine is substituted with a functionality selected from the group consisting of fluoride, chloride, bromide, iodide, nitro, hydroxyl, ether, sulfide, disulfide, sulfoxide and sulfone.

18. The method of claim 17 wherein said functionality is chloride.

19. The method of any one of claims 15 to 18 wherein said monoamine includes octadecylamine.

20. The method of any one of claims 15 to 19 wherein said biguanide is a polymeric biguanide having recurring units of the general formula: 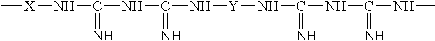
where the organic radicals represented by X and Y are the same or different organic bridging groups and conveniently are the same or different C₂ to C₁₄₀ aliphatic, cycloaliphatic, heterocyclic, aryl, alkaryl, aralkyl or oxyalkylene radicals.

21. The method of claim 20 wherein at least one of X and Y is a C₄ to C₁₆ polymethylene radical.

22. The method of any one of claims 15 to 21 wherein said biguanide is polyhexamethylene biguanide.
